(19) United States
(12) Patent Application Publication
(10) Pub. No.: US 2020/0088731 A1
(43) Pub. Date: Mar. 19, 2020

(54) Method for Detecting HER2-Positive Cancer Cells

(71) Applicant: Hitachi Chemical Company, Ltd., Tokyo (JP)

(72) Inventors: Seita Nakamura, Tokyo (JP); Satomi Yagi, Tokyo (JP); Katsuya Endo, Tokyo (JP); Masayuki Higuchi, Tokyo (JP)

(73) Assignee: Hitachi Chemical Company, Ltd., Tokyo (JP)

(21) Appl. No.: 16/473,051

(22) PCT Filed: Dec. 22, 2016
(86) PCT No.: PCT/JP2016/088503

§ 371 Date: Jun. 24, 2019

(51) Int. Cl. G01N 33/574; G01N 33/533

(52) U.S. Cl. CPC G01N 33/57415; G01N 33/533

(57) ABSTRACT

A method for detecting HER2-positive cancer cells, comprising steps of: (a) fixing and then permeabilizing cells; (b) contacting a primary antibody that recognizes an intracellular epitope of HER2 with the cells and then contacting a secondary antibody that recognizes the primary antibody and is labeled with a fluorescent dye; and (c) irradiating the cells with excitation light for the fluorescent dye and detecting fluorescence emitted from the cells is provided. According to such a method, HER2-positive cancer cells can be detected with high sensitivity.

### TECHNICAL FIELD

The present invention relates to a method for detecting HER2-positive cancer cells.

### BACKGROUND ART

A “molecular targeted therapy” is known as one of the methods for treating cancer. Since the molecular targeted therapy specifically acts on a cancer-related substance (marker protein) overexpressed in cancer cells, the side effect tends to be little. Examples of the marker protein include a receptor tyrosine kinase called HER2 (Human Epidermal Growth Factor Receptor 2), and a molecular targeted drug “trastuzumab”, which targets HER2, has been put to practical use. It has been reported that trastuzumab is dramatically effective in patients having cancer cells in which HER2 is overexpressed (HER2-positive). The detection of the overexpression of HER2 in cancer cells of patients is highly required to confirm the effectiveness of an anticancer agent such as trastuzumab targeting HER2 before the anticancer agent is actually administered.

HER2-positive cancer cells can be detected by reacting an antibody that recognizes HER2 and is fluorescently labeled with cells and detecting this fluorescence (for example, Patent Literature 1).

### CITATION LIST

### Patent Literature

Patent Literature 1: Japanese Unexamined Patent Publication No. 2008-116466

### SUMMARY OF INVENTION

### Technical Problem

When the present inventors fluorescently labeled the HER2-positive cancer cells and attempted detecting them, there were cases where fluorescence indicating HER2 was not detected or the fluorescent brightness was weak even when the fluorescence was detected, so that HER2-positive cancer cells could not be detected.

### Solution to Problem

The present inventors have earnestly investigated in view of such a situation, and consequently found that by fluorescently labeling HER2-positive cancer cells using a specific antibody after fixing and permeabilizing cells, the HER2-positive cancer cells can be detected with high sensitivity, and completed the present invention.

That is, the present invention provides a method for detecting HER2-positive cancer cells, including steps of: (a) fixing and then permeabilizing cells; (b) contacting a primary antibody that recognizes an intracellular epitope of HER2 with the cells and then contacting a secondary antibody that recognizes the primary antibody and is labeled with a fluorescent dye; and (c) irradiating the cells with excitation light for the fluorescent dye and detecting fluorescence emitted from the cells.

The cells may be cells collected from a blood sample. The above-mentioned fluorescent dye may be a first fluorescent dye, and steps of: (x1) contacting a primary antibody that recognizes a marker protein for white blood cells with the cells and then contacting a secondary antibody that recognizes the primary antibody and is labeled with a second fluorescent dye; (x2) contacting an antibody that recognizes a marker protein for epithelial cells and is labeled with a third fluorescent dye with the cells; and (x3) labeling nuclei of the cells with a fourth fluorescent dye may be further performed in any order at any stage before step (c). In this case, in step (c), the cells are irradiated with the respective excitation lights for the first, second, third and fourth fluorescent dyes, and the respective fluorescences of the first, second, third and fourth fluorescent dyes emitted from the cells are detected.

The primary antibody that recognizes HER2 may be derived from a clone selected from the group consisting of 4B5, EP1045Y and K.929.9. The cells may be cells captured on a filter by filtering a blood sample through the filter. Step (x1) may be performed before step (a), and step (x2) and step (x3) may be performed after step (a). The marker protein for white blood cells may be CD45. The marker protein for epithelial cells may be cytokeratin. The HER2-positive cancer cells may be derived from breast cancer.

### Advantageous Effects of Invention

According to the present invention, HER2-positive cancer cells can be detected with high sensitivity.

### BRIEF DESCRIPTION OF DRAWINGS

FIG. 1 is a perspective view showing one embodiment of a cell-capturing cartridge.

FIG. 2 is a sectional view taken from line in FIG. 1.

FIG. 3 shows images of cells fluorescently labeled in Test Example 1.

FIG. 4 shows images of cells fluorescently labeled in Test Example 1.

FIG. 5 shows images of cells fluorescently labeled in Test Example 2.

### DESCRIPTION OF EMBODIMENTS

A method for detecting HER2-positive cancer cells of the present invention comprises steps of: (a) fixing and then permeabilizing cells; (b) contacting a primary antibody that recognizes an intracellular epitope of HER2 with the cells and then contacting a secondary antibody that recognizes the primary antibody and is labeled with a fluorescent dye; and (c) irradiating the cells with excitation light for the fluorescent dye and detecting fluorescence emitted from the cells. According to this method, HER2-positive cancer cells derived from, for example, breast cancer, lung cancer, stomach cancer, salivary gland cancer or ovarian cancer can be detected. “Contacting” a substance with cells may be performed, for example, by immersing the cells in the substance or a solution of the substance.

In step (a), cells which may comprise HER2-positive cancer cells are first fixed. The cells may be fixed by contacting a well-known fixing agent such as formaldehyde with the cells. By fixing the cells, the decomposition or aggregation of the cells may be further reduced.

The fixed cells are then permeabilized. The cells may be permeabilized by contacting a well-known permeabilizing agent with the cells. As the permeabilizing agent, for example, poly(oxyethylene)octylphenyl ether may be used.

The cells may be washed after step (a). The washing step is performed, for example, by contacting a washing solution comprising a known buffer solution such as phosphate buffered saline (PBS) with the cells. Additives such as bovine serum albumin (BSA) and ethylenediaminetetraacetic acid (EDTA) may be contained in the washing solution. Washing may be performed not only after step (a) but also after each step, as required. The washing step may be performed before permeabilizing and after the cells are fixed.

In step (b), a primary antibody that recognizes an intracellular epitope of HER2 is contacted with cells, and a secondary antibody that recognizes the primary antibody and is labeled with a fluorescent dye (first fluorescent dye) is contacted. HER2 is fluorescently labeled by this step. HER2 may be fluorescently labeled in two stages as mentioned above, or may be labeled in one stage. That is, HER2 may be fluorescently labeled in one stage by contacting an antibody that recognizes an intracellular epitope of HER2 and is labeled with the fluorescent dye with the cells.

It is preferable that the primary antibody that recognizes an intracellular epitope of HER2 or the antibody that recognizes an intracellular epitope of HER2 and is labeled with a first fluorescent dye is derived from a clone selected from the group consisting of K.929.9, 4B5, EP1045Y, D8F12, 6B12, HRB2/451, 29D8, 4F10, 3B5 and CB11, and it is more preferable that it is derived from a clone selected from the group consisting of K.929.9, 4B5 and EP1045Y. HER2-positive cancer cells can be detected with higher sensitivity using an antibody derived from these clones. The antibodies derived from K.929.9, 4B5 or EP1045Y are all anti-HER2 rabbit monoclonal antibodies.

The fluorescent dye (first fluorescent dye) is not particularly limited as long as it is a fluorescent dye usually used for the fluorescent labeling of antibodies. The first fluorescent dye is, for example, Alexa Fluor (registered trademark) 647.

Finally, in step (c), the cells are irradiated with the excitation light for the fluorescent dye, and the fluorescence emitted from the cells is detected. Cells from which the fluorescence of the fluorescent dye (first fluorescent dye) is detected (Positive) are identified as HER2-positive cancer cells.

The analysis of DNA, RNA or protein may then be performed on the detected HER2-positive cancer cells. Analysis, for example, using a sequencer, a next-generation sequencer, a DNA chip, a microarray, comparative genomic hybridization, fluorescence in situ hybridization, digital PCR, quantitative reverse transcription PCR, ELISA, Western blotting, TOF-MS, MALDI-MS, a Raman spectroscopic spectrum, chromatography, X-ray crystal analysis, two-dimensional electrophoresis, nuclear magnetic resonance spectroscopy, a flow cytometer (FCM) or the like may be performed on the detected HER2-positive cancer cells.

In the detecting method of the present invention, cells used may be collected from blood or lymph, or may be collected from tissue. Cancer cells called Circulating Tumor Cells (hereinafter also called “CTCs”) which circulate through the blood vessel and the lymph vessel throughout the body may be present in the blood of cancer patients. Detecting CTCs overexpressing HER2 is effective in treating cancer effectively and at an early stage.

A method for detecting HER2-positive cancer cells in a blood sample according to one embodiment of the present invention will be described hereinafter. In this embodiment, the above-mentioned steps (a) to (c) are performed on cells collected from blood.

As the blood sample, blood collected from a subject may be used as it is, and blood diluted with a buffer solution such as PBS or a suitable medium may also be used. Additives such as an anticoagulant and a fixing agent usually added to blood samples may be added to the blood sample.

Cells may be collected from blood, for example, by filtering a blood sample through a filter and capturing the cells in the blood sample on the filter. When collecting the cells in blood with a filter, detection of HER2-positive cancer cells may be conducted on the filter. That is, all the steps (including optional steps described below) in the present invention may be performed on the cells captured on the filter. “Capturing” means filtering a liquid comprising cells with a filter and leaving the cells on the filter.

Contacting a reaction solution or a washing solution with cells may be performed by filtering these solutions with the filter. From the viewpoint of minimizing damage to the cells at the time of filtration, the flow rate of the solution is preferably 50 μL/min to 3000 more preferably 100 μL/min to 1000 and further preferably 200 μL/min to 600 μL/min.

The filter is not particularly limited as long as it is a filter that is able to capture CTCs existing in blood samples, and a conventionally known filter may be used. The filter may be, for example, a metal filter, and has through holes with a pore size of preferably 5 μm to 15 μm, more preferably 6 μm to 12 μm, further preferably 7 μm to 10 μm. The pore size of the through holes refers to the maximum value of the diameter of a sphere that is able to pass a through hole. Since white blood cells among cells contained in blood have a similar diameter as CTCs, white blood cells are captured together with CTCs on the filter.

In the blood of cancer patients, many other cells such as HER2-negative CTCs and white blood cells exist besides HER2-positive CTCs. Therefore, there are cases where the antibody that recognizes HER2 binds to HER2-negative cells and fluorescence indicating HER2 is observed from the HER2-negative cells (false positive). From the viewpoint of reducing such false positives and detecting HER2-positive cancer cells more certainly, it is preferable to further perform the following steps (x1) to (x3).

In step (x1), a primary antibody that recognizes a marker protein for white blood cells is contacted with cells, and a secondary antibody that recognizes the primary antibody and is labeled with a second fluorescent dye is then contacted. The white blood cells are fluorescently labeled by this step. The white blood cells may be fluorescently labeled in two stages as mentioned above, or may be labeled in one stage. That is, white blood cells may be fluorescently labeled in one stage by contacting an antibody that recognizes the marker protein for white blood cells and is labeled with the second fluorescent dye with cells.

The marker protein for white blood cells is, for example, CD45, which is expressed on all hematopoietic stem cells.

The primary antibody that recognizes the marker protein for white blood cells, the secondary antibody labeled with the second fluorescent dye and the antibody that recognizes the marker protein for white blood cells and is labeled with the second fluorescent dye are not particularly limited, and they may be polyclonal antibodies or monoclonal antibodies. Animals from which the antibodies are derived are not particularly limited as long as an animal from which the primary antibody is derived and an animal from which the secondary antibody is derived are different.

The second fluorescent dye is not particularly limited as long as it is a fluorescent dye usually used for the fluorescent labeling of antibodies. The second fluorescent dye is a fluorescent dye different from the first, third and fourth fluorescent dyes. Since the fluorescent dyes have different fluorescence wavelengths, they can be distinguished. The second fluorescent dye is, for example, Alexa Fluor (registered trademark) 594.

In step (x2), an antibody that recognizes a marker protein for epithelial cells and is labeled with a third fluorescent dye is contacted with the cells. CTCs are fluorescently labeled by this step.

Examples of the marker protein for epithelial cells include cytokeratin, epithelial cell adhesion molecules (EpCAMs), CD146 and CD176, and cytokeratin is preferable. Since CTCs are derived from epithelial cells, they have these marker proteins for epithelial cells.

The third fluorescent dye is not particularly limited as long as it is fluorescent dye usually used for the fluorescent labeling of antibodies. The third fluorescent dye is fluorescein such as fluorescein isothiocyanate (FITC).

The antibody that recognizes the marker protein for epithelial cells is not be particularly limited, and may be a polyclonal antibody or a monoclonal antibody. An animal from which the antibody that recognizes the marker protein for epithelial cells is derived is not limited.

In step (x3), the nuclei of the cells are labeled with a fourth fluorescent dye. The fourth fluorescent dye which labels nuclei is not particularly limited as long as it is a fluorescent dye which binds to nucleic acid, and a fluorescent dye usually used for labeling nuclei fluorescently may be used. Examples of the fourth fluorescent dye include 4′,6-diamidino-2-phenylindole (DAPI) and 2′-(4-ethoxy-phenyl)-5-(4-methyl-1-piperazinyl)-2,5′-bi-1H-benzimidazole trihydrochloride (Hoechst 33342).

Steps (x1) to (x3) may be performed at any stage before step (c), and may be performed in any order. For example, the steps may be performed in the order of step (x1), step (a), step (b), step (x2), step (x3) and step (c); and step (x2) and step (x3) may be performed simultaneously.

When these optional steps are performed, in step (c), the cells are irradiated with the respective excitation lights for the first, second, third and fourth fluorescent dyes, and the respective fluorescences of the first, second, third and fourth fluorescent dyes emitted from the cells are detected. HER2 is labeled with the first, third and fourth fluorescent dyes. Therefore, cells from which fluorescence of the second fluorescent dye is not detected (negative) and the fluorescences by the first, third and fourth fluorescent dyes are detected (positive) are identified as HER2-positive CTCs.

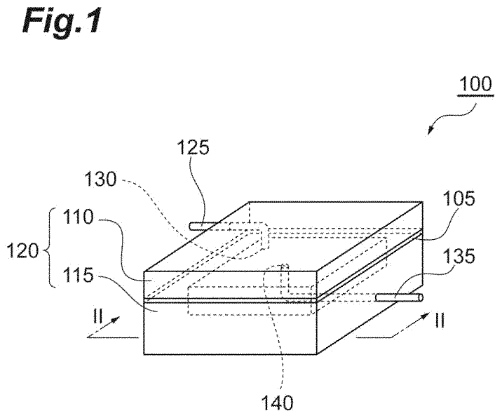

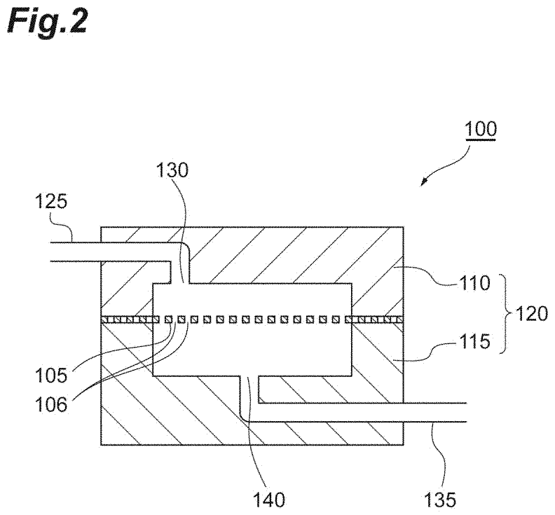

When detecting HER2-positive CTCs in a blood sample by the above-mentioned method, for example, a cartridge shown in FIG. 1 and FIG. 2 may be used. A method for detecting HER2-positive cancer cells in blood samples using a cartridge according to one embodiment of the present invention will be described hereinafter. Unless otherwise specified, the details of the steps and the order of the steps are as described in the above-mentioned embodiment.

A CTC-capturing cartridge (cartridge) 100 shown in FIG. 1 and FIG. 2 comprises: a case 120 having an inlet port 130 to which an inlet pipe 125 into which liquid flows is connected and an outlet port 140 to which an outlet pipe 135 out of which liquid flows is connected; and a filter 105. A filter 105 is fixed by the case 120 consisting of an upper member 110 and a lower member 115. Blood samples, a washing solution and other reaction solutions are introduced into the case 120 through the inlet pipe 125, pass through the filter 105, and are discharged outside from the outlet pipe 135. Such a flow of liquid may be generated, for example, by connecting a pump upstream of the inlet pipe 125 or downstream of the outlet pipe 135. A cock may be provided upstream of the inlet pipe 125 and/or downstream of the outlet pipe 135 to regulate the flow of liquid.

Firstly, a blood sample is introduced into the cartridge 100 from the inlet pipe 125 to filter the blood sample through the filter 105. White blood cells and CTCs in the blood sample cannot pass through through holes 106 of the filter 105 and remain on the surface of the filter 105. The other components of the blood sample pass through the through holes 106 and are discharged out of the cartridge 100. Next, a washing solution may be passed through the filter 105 to wash the filter 105. The filter 105 may also be washed, as required, after each of the following steps.

After cells are captured on the filter 105, a reaction solution comprising a fixing agent and then a reaction solution comprising a permeabilizing agent are introduced into the cartridge 100 and retained in the cartridge 100 for predetermined time, respectively, so that the cells are reacted with the fixing agent and the permeabilizing agent, respectively (step (a)). In the same manner, a reaction solution comprising a primary antibody that recognizes an intracellular epitope of HER2, and then a reaction solution comprising a secondary antibody that recognizes the primary antibody and is labeled with a fluorescent dye are reacted with the cells captured on the filter 105, respectively (step (b)). Lastly, the cartridge 100 is irradiated with the excitation light for fluorescent dye using a fluorescence microscope, and fluorescence emitted from the cells captured on the filter 105 is detected (step (c)). Fluorescence may be detected, for example, by observing the cartridge 100 from the upper surface of the cartridge 100 in the vertical direction and processing the fluorescence observation image. Steps (x1) to (X3) maybe optionally performed as described in the above-mentioned embodiment.

### EXAMPLES

### Test Example 1

### Example 1

SKBR3 (HER2-positive), a cell strain derived from human breast cancer, in a culture flask were cultured in a carbon dioxide incubator at 37° C. Trypsin-EDTA at a concentration of 0.25% was added to the culture flask to dissociate from the flask, the cultured cells adhering to the flask. The dissociated cells were counted using an erythrocytometer and a phase microscope, 1.0×10⁶ cells were added to a centrifugal tube and suspended in a PBS solution comprising 0.5% BSA and 2 mM EDTA (hereinafter called “a washing solution”).

The centrifugal tube was centrifuged at a centrifugal force 400×g, and the supernatant was removed. 1.25 mL of a reaction solution comprising an anti-human CD45 mouse monoclonal antibody (clone: 2D1) was added to the pellet in the centrifugal tube, and the mixture was reacted at room temperature for 30 minutes. The reaction solution was removed by centrifugation, and 1.40 mL of the washing solution was then added to wash the pellet. The washing solution was removed by centrifugation, 1.25 mL of a reaction solution comprising an Alexa Fluor (registered trademark) 594-labeled anti-mouse IgG goat polyclonal antibody was then added, and the mixture was reacted at room temperature for 30 minutes. The reaction solution was removed by centrifugation, and 1.40 mL of the washing solution was then added to wash the pellet.

The washing solution was removed by centrifugation, 1.25 mL of a PBS solution comprising 0.5% by mass to 4% by mass of formaldehyde was then added, and the mixture was reacted at room temperature for 10 minutes to fix the cells. The reaction solution was removed by centrifugation, and 1.40 mL of the washing solution was then added to wash the pellet.

The washing solution was removed by centrifugation, 1.25 mL of a PBS solution comprising 0.05% by mass to 0.1% by mass of Triton X-100 (produced by Sigma-Aldrich Co. LLC.) was then added, and the mixture was reacted at room temperature for 10 minutes to permeabilize the cells. The reaction solution was removed by centrifugation, and 1.40 mL of the washing solution was then added to wash the pellet.

The washing solution was removed by centrifugation, 1.25 mL of a reaction solution comprising an anti-human HER2 rabbit monoclonal antibody (clone: 4B5) was then added, and the mixture was reacted at room temperature for 30 minutes. The reaction solution was removed by centrifugation, and 1.40 mL of the washing solution was then added to wash the pellet. The washing solution was removed by centrifugation, 1.25 mL of a reaction solution comprising an Alexa Fluor (registered trademark) 647-labeled anti-rabbit IgG goat polyclonal antibody was then added, and the mixture was reacted at room temperature for 30 minutes. The reaction solution was removed by centrifugation, and 1.40 mL of the washing solution was then added to wash the pellet.

The washing solution was removed by centrifugation, 1.25 mL of a reaction solution comprising a FITC-labeled anti-human cytokeratin mouse monoclonal antibody (clone: mixture of CK3/6H5/AE1/AE3), DAPI and a washing solution was then added, and the mixture was reacted at room temperature for 30 minutes. The reaction solution was removed by centrifugation, and 3.00 mL of the washing solution was then added to wash the pellet and obtain a cell suspension.

10 μL of the obtained cell suspension was dropped on a slide glass and covered with a cover glass. The slide glass was placed under the fluorescence microscope, and the fluorescent dyes (FITC, Alexa Fluor 594, Alexa Fluor 647 and DAPI) on the cells were excited respectively using fluorescence mirror units. Fluorescence emitted from the fluorescent dyes was photographed, and each fluorescent brightness was analyzed from the obtained images using an image analysis software Columbus (produced by PerkinElmer Japan Co., Ltd.).

### Example 2

An experiment was performed in the same way as in Example 1 to analyze the fluorescent brightness of the cells, except that the clone of the anti-human HER2 rabbit monoclonal antibody was changed to EP1045Y.

### Example 3

An experiment was performed in the same way as in Example 1 to analyze the fluorescent brightness of the cells, except that the clone of the anti-human HER2 rabbit monoclonal antibody was changed to K.929.9.

### Comparative Example 1

An experiment was performed in the same way as in Example 1 to analyze the fluorescent brightness of the cells, except that the clone of the anti-human HER2 rabbit monoclonal antibody was changed to SP3. The above-mentioned antibody derived from the clone SP3 is an antibody that recognizes an extracellular epitope of HER2.

### Comparative Example 2

An experiment was performed in the same way as Example 3 to analyze the fluorescent brightness of the cells, except that HER2 was fluorescently labeled before the cells were fixed and permeabilized.

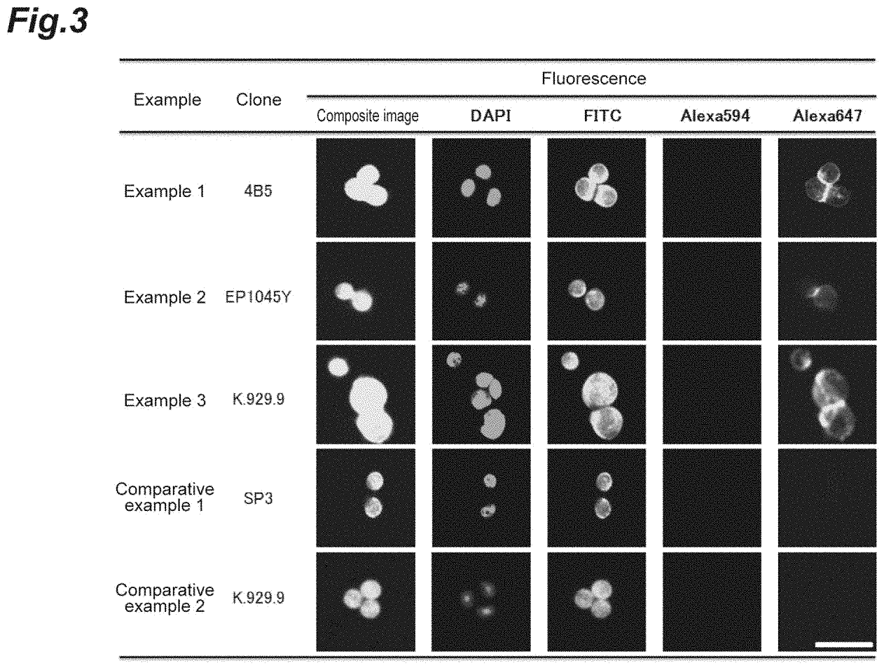

The results of Examples 1 to 3 and Comparative Examples 1 and 2 are shown in Table 1 and FIG. 3. Since SKBR3 is a cell strain of HER2-positive cancer, fluorescence images that are DAPI (nuclei)-positive, FITC (cytokeratin)-positive, Alexa Fluor 594 (CD45)-negative and Alexa Fluor 647 (HER2)-positive should be obtained. Here, “positive” and “negative” are determined by the intensity of fluorescent brightness (RFU). In Examples 1 to 3, since the antibody that recognizes an intracellular epitope of HER2 was used and the cells were fixed and permeabilized before the fluorescent labeling of HER2, the fluorescent brightness of Alexa Fluor 647 was markedly higher than that in Comparative Example 1 and Comparative Example 2, and SKBR3, which were HER2-positive, could be detected with high sensitivity. The fluorescent brightness of Alexa Fluor 647 in Comparative Example 1 in which an antibody that recognizes an extracellular epitope of HER2 and that in Comparative Example 2 in which the cells were fixed and permeabilized after the fluorescent labeling of HER2 were both low, and SKBR3 could not be detected.

TABLE 1

| | Cell | Anti-HER2 | Fluorescent brightness (RFU) | | | |
|---|---|---|---|---|---|---|
| Example | strain | antibody clone | DAPI | FITC | Alexa594 | Alexa647 |
| Example 1 | SKBR3 | 4B5 | 10325 | 145923 | 347 | 21239 |
| Example 2 | SKBR3 | EP1045Y | 10545 | 167011 | 381 | 23891 |
| Example 3 | SKBR3 | K.929.9 | 10435 | 162474 | 384 | 24740 |
| Comparative | SKBR3 | SP3 | 9113 | 177806 | 139 | 896 |
| Example 1 | | | | | | |
| Comparative | SKBR3 | K.929.9 | 10526 | 161002 | 368 | 171 |
| Example 2 | | | | | | |

### Example 4

An experiment was performed in the same way as in Example 1 to analyze the fluorescent brightness of the cells, except that the cell strain derived from human breast cancer was changed to MDA-MB-231 (HER2-negative).

### Example 5

An experiment was performed in the same way as in Example 2 to analyze the fluorescent brightness of the cells, except that the cell strain was changed to MDA-MB-231.

### Example 6

An experiment was performed in the same way as in Example 3 to analyze the fluorescent brightness of the cells, except that the cell strain was changed to MDA-MB-231.

### Comparative Example 3

An experiment was performed in the same way as in Comparative Example 1 to analyze the fluorescent brightness of the cells, except that the cell strain was changed to MDA-MB-231.

### Comparative Example 4

An experiment was performed in the same way as in Comparative Example 2 to analyze the fluorescent brightness of the cells, except that the cell strain was changed to MDA-MB-231.

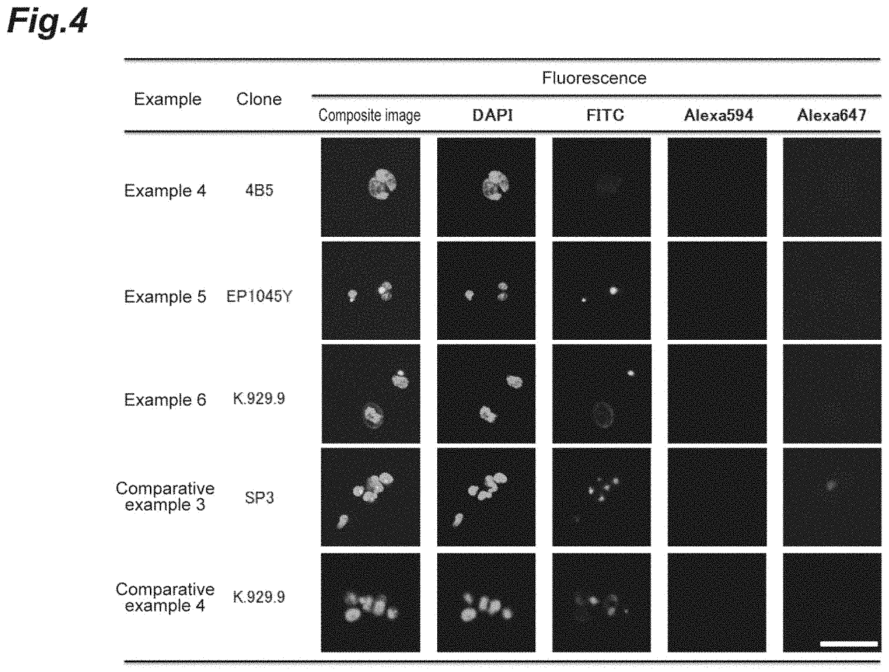

The results of Examples 4 to 6 and Comparative Examples 3 and 4 are shown in Table 2 and FIG. 4. Since MDA-MB-231 is a cell strain of HER2-negative cancer, fluorescence images that are DAPI (nuclei)-positive, FITC (cytokeratin)-positive, Alexa Fluor 594 (CD45)-negative and Alexa Fluor 647 (HER2)-negative should be obtained. In any of the Examples and the Comparative Examples, the fluorescent brightness of Alexa Fluor 647 was weak as expected.

TABLE 2

| | Cell | Anti-HER2 | Fluorescent brightness (RFU) | | | |
|---|---|---|---|---|---|---|
| Example | strain | antibody clone | DAPI | FITC | Alexa594 | Alexa647 |
| Example 4 | MDA-MB-231 | 4B5 | 6076 | 26527 | 105 | 319 |
| Example 5 | MDA-MB-231 | EP1045Y | 8116 | 29355 | 107 | 615 |
| Example 6 | MDA-MB-231 | K.929.9 | 7067 | 23584 | 106 | 525 |
| Comparative | MDA-MB-231 | SP3 | 6947 | 24252 | 106 | 450 |
| Example 3 | | | | | | |
| Comparative | MDA-MB-231 | K.929.9 | 7096 | 27365 | 105 | 505 |
| Example 4 | | | | | | |

### Test Example 2

### Example 7

SKBR3 (HER2-positive), a cell strain derived from human breast cancer, in a culture flask were cultured in a carbon dioxide incubator at 37° C. Trypsin-EDTA at a concentration of 0.25% was added to the culture flask to dissociate from the flask, the cultured cells adhering to the flask. The dissociated cells were counted using the erythrocytometer and the phase microscope. 10000 cells were added to the blood from a healthy person collected in a blood collection tube to prepare a blood sample. A Cell-Free DNA blood collection tube manufactured by Streck, Inc. was used as the blood collection tube.

HER2-positive cancer cells in the above-mentioned blood sample was detected as follows using a CTC-capturing cartridge (cartridge) in which a metal filter (film area 6 mm×6 mm, film thickness 18 μm) which was a thin film having multiple through holes with a major axis of 100 μm and a minor axis of 8 μm was incorporated. The CTC-capturing cartridge corresponds to the cartridge 100 described in the above-mentioned embodiment. A CTC-capturing device comprises a reservoir in which a blood sample and other reaction solutions are introduced.

The cartridge was first filled with a PBS solution comprising 0.5% BSA and 2 mM EDTA (hereinafter called “a washing solution”). The reservoir was charged with 7 mL of the washing solution, and 3 mL of the above-mentioned blood sample was added under the washing solution so that the blood sample and the washing solution formed layers. The CTC-capturing device was started, the blood sample and the washing solution in the reservoir were introduced into the cartridge at a flow rate of 600 μL/min, and white blood cells in the blood sample were captured on the filter. The washing solution was introduced into the cartridge and blood components remaining on the filter was washed.

1.25 mL of a reaction solution comprising an anti-human CD45 mouse monoclonal antibody (clone: 2D1) was introduced into the cartridge at a flow rate of 200 μL/min, and was reacted at room temperature for 30 minutes. 1.40 mL of the washing solution was introduced into the cartridge at a flow rate of 400 μL/min to discharge the above-mentioned reaction solution in the cartridge. 1.25 mL of a reaction solution comprising an Alexa Fluor (registered trademark) 594-labeled anti-mouse IgG goat polyclonal antibody was introduced into the cartridge at a flow rate of 400 μL/min, and was reacted at room temperature for 30 minutes. 1.40 mL of the washing solution was introduced into the cartridge at a flow rate of 400 μL/min to discharge the above-mentioned reaction solution in the cartridge.

1.25 mL of a PBS solution comprising 0.5% by mass to 4% by mass of formaldehyde was introduced into the cartridge at a flow rate of 400 μL/min and was reacted at room temperature for 10 minutes to fix the cells. 1.40 mL of the washing solution was introduced into the cartridge at a flow rate of 400 μL/min to discharge the above-mentioned reaction solution in the cartridge.

1.25 mL of a PBS solution comprising 0.05% by mass to 0.1% by mass of Triton X-100 (produced by Sigma-Aldrich Co. LLC.) was introduced into the cartridge at a flow rate of 400 μL/min and was reacted at room temperature for 10 minutes to permeabilize the cells. 1.40 mL of the washing solution was introduced into the cartridge at a flow rate of 400 μL/min to discharge the above-mentioned reaction solution in the cartridge.

1.25 mL of a reaction solution comprising an anti-human HER2 rabbit monoclonal antibody (clone: K.929.9) was introduced into the cartridge at a flow rate of 400 μL/min, and was reacted at room temperature for 30 minutes. 1.40 mL of the washing solution was introduced into the cartridge at a flow rate of 400 μL/min to discharge the above-mentioned reaction solution in the cartridge. 1.25 mL of a reaction solution comprising an Alexa Fluor (registered trademark) 647-labeled anti-rabbit IgG goat polyclonal antibody was introduced into the cartridge at a flow rate of 400 μL/min, and was reacted at room temperature for 30 minutes. 1.40 mL of the washing solution was introduced into the cartridge at a flow rate of 400 μL/min to discharge the above-mentioned reaction solution in the cartridge.

1.25 mL of a reaction solution comprising an FITC-labeled anti-human cytokeratin mouse monoclonal antibody (clone: mixture of CK3/6H5/AE1/AE3), DAPI and the washing solution was introduced into the cartridge at 400 μL/min, and was reacted at room temperature for 30 minutes. 3.00 mL of the washing solution was introduced into the cartridge at a flow rate of 400 μL/min to discharge the above-mentioned reaction solution in the cartridge. Subsequently, the cartridge was removed from the CTC capturing device.

The cartridge was placed under a fluorescence microscope. The fluorescent dyes (FITC, Alexa Fluor 594, Alexa Fluor 647 and DAPI) on the cells were excited using fluorescence mirror units. Fluorescence emitted from each fluorescent dye was photographed, and fluorescent brightness of the each was analyzed from the obtained image using an image analysis software Columbus (produced by PerkinElmer Japan Co., Ltd.). More specifically, firstly, the nucleus region of each cell was recognized by the fluorescence of DAPI, and subsequently, the cytoplasm region of each cell was recognized by the fluorescence of FITC, Alexa Fluor 594 and Alexa Fluor 647 in the periphery of the recognized nucleus region. The average brightness in the recognized nucleus region was determined as the fluorescent brightness of DAPI, and the average brightness in the recognized cytoplasm region was determined as the fluorescent brightness of the other fluorescent dyes.

### Example 8

An experiment was performed in the same way as in Example 7 to analyze the fluorescent brightness of the cells, except that the cell strain derived from human breast cancer was changed to MDA-MB-231 (HER2-negative).

### Example 9

An experiment was performed in the same way as in Example 7 to analyze the fluorescent brightness of the cells, except that the blood collection tube was changed to a blood collection tube comprising EDTA-2K (dipotassium ethylenediaminetetraacetate) manufactured by Becton, Dickinson and Company.

### Example 10

An experiment was performed in the same way as in Example 9 to analyze the fluorescent brightness of the cells, except that the cell strain derived from human breast cancer was changed to ZR-75-1 (HER2-positive).

### Example 11

An experiment was performed in the same way as in Example 9 to analyze the fluorescent brightness of the cells, except that the cell strain derived from human breast cancer was changed to MDA-MB-231 (HER2-negative).

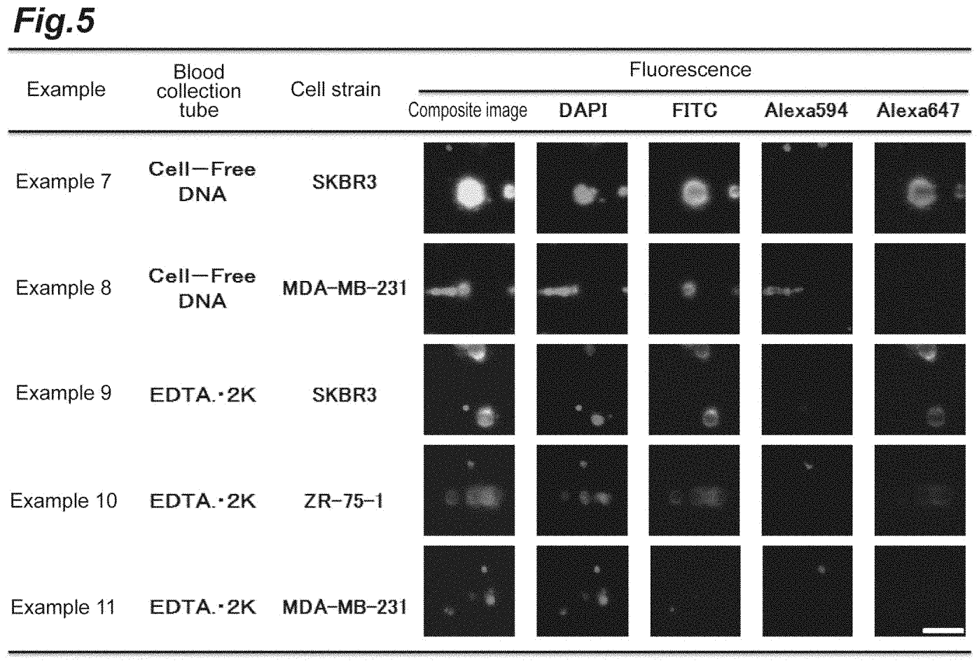

The results of Examples 7 to 11 are shown in Table 3. The results of Examples 9 to 11 are also shown in FIG. 5. Since SKBR3 was a cell strain which overexpressed HER2, the fluorescent brightness of Alexa Fluor 647 was the most intense (Examples 7 and 9). Although ZR-75-1 overexpressed HER2, the level of expression was not as high as that of SKBR3, and thus, the fluorescent brightness was not as high as that of SKBR3 (Example 10). Since MDA-MB-231 was a HER2-negative cell strain, the fluorescent brightness of Alexa Fluor 647 was low (Examples 8 and 11). According to the method of the present invention, it has been shown that HER2-positive cancer cells can be detected from the fluorescent brightness dependent on the amount of HER2 expressed.

TABLE 3

| | | Fluorescent brightness (pixel) | | | |
|---|---|---|---|---|---|
| Example | Cell strain | DAPI | FITC | Alexa594 | Alexa647 |
| Example 9 | SKBR3 | 56.5 | 18.7 | 3.8 | 16.5 |
| Example 10 | ZR-75-1 | 41.4 | 9.2 | 3.6 | 5.8 |
| Example 11 | MDA-MB-231 | 40.7 | 2.9 | 3.5 | 2.1 |

### REFERENCE SIGNS LIST

100: CTC-capturing cartridge, 105: filter, 106: through holes, 110: upper member, 115: lower member, 120: case, 125: inlet pipe, 130: inlet port, 135: outlet pipe, 140: outlet port.

1. A method for detecting HER2-positive cancer cells, comprising steps of:
(a) fixing and then permeabilizing cells;
(b) contacting a primary antibody that recognizes an intracellular epitope of HER2 with the cells and then contacting a secondary antibody that recognizes the primary antibody and is labeled with a fluorescent dye; and
(c) irradiating the cells with excitation light for the fluorescent dye and detecting fluorescence emitted from the cells.

2. The method according to claim 1,
wherein the cells are cells collected from a blood sample,
wherein the fluorescent dye is a first fluorescent dye,
wherein steps of:
(x1) contacting a primary antibody that recognizes a marker protein for white blood cells with the cells and then contacting a secondary antibody that recognizes the primary antibody and is labeled with a second fluorescent dye;
(x2) contacting an antibody that recognizes a marker protein for epithelial cells and is labeled with a third fluorescent dye with the cells; and
(x3) labeling nuclei of the cells with a fourth fluorescent dye are further performed in any order at any stage before step (c), and
wherein in step (c), the cells are irradiated with the respective excitation lights for the first, second, third and fourth fluorescent dyes, and the respective fluorescences of the first, second, third and fourth fluorescent dyes emitted from the cells are detected.

3. The method according to claim 2, wherein the primary antibody that recognizes HER2 is derived from a clone selected from the group consisting of 4B5, EP1045Y and K.929.9.

4. The method according to claim 2 or 3, wherein the cells are cells captured on a filter by filtering a blood sample through the filter.

5. The method according to any one of claims 2 to 4, wherein
step (x1) is performed before step (a), and
step (x2) and step (x3) are performed after step (a).

6. The method according to any one of claims 2 to 4, wherein the marker protein for white blood cells is CD45.

7. The method according to any one of claims 2 to 5, wherein the marker protein for epithelial cells is cytokeratin.

8. The method according to any one of claims 2 to 6, wherein the HER2-positive cancer cells are derived from breast cancer.
